(19) United States
(12) Patent Application Publication
(10) Pub. No.: US 2013/0079285 A1
(43) Pub. Date: Mar. 28, 2013

(54) SOLUBILIZED COLLAGEN FIBERS AND METHOD FOR PRODUCING THE SAME

(71) Applicants: Jun-ichi Satoh, Yamagata-shi (JP); Kazutaka Endoh, Yamagata-shi (JP); Susumu Suzuki, Yamagata-shi (JP)

(72) Inventors: Jun-ichi Satoh, Yamagata-shi (JP); Kazutaka Endoh, Yamagata-shi (JP); Susumu Suzuki, Yamagata-shi (JP)

(73) Assignee: MIDORI HOKUYO CO., LTD., Yamagata-shi, Yamagata (JP)

(21) Appl. No.: 13/700,623

(22) PCT Filed: May 30, 2011
(86) PCT No.: PCT/JP2011/062864

§ 371 Date: Nov. 28, 2012

(30) Foreign Application Priority Data
May 28, 2010 (JP) 2010-122822

(51) Int. Cl. A61K 38/39

(52) U.S. Cl. CPC A61K 38/39
USPC 514/17.2

(57) ABSTRACT

Provided are novel solubilized collagen fibers with which solubilized collagen can be obtained by instantaneous uniform dissolution in water at the time of use. The solubilized collagen fibers are formed from a solubilized collagen solid content of 66 to 87 wt %, buffer salt content of 2 to 6 wt %, water content of 10 to 22 wt %, and a residual hydrophilic organic solvent content of a trace amount of up to 6 wt % (totaling 100 wt %). The solubilized collagen fibers have an average fineness of 3 to 10 dtx, an isoionic point of 4.5 to 5.0, a water content of 10 to 22 wt %, and residual hydrophilic organic solvent content of a trace amount of up to 6.0 wt %. The solubilized collagen fibers are present uniformly in the direction of fiber length. The buffer salt is selected from sodium citrate, sodium lactate, and sodium phosphate.

### TECHNICAL FIELD

The present invention relates to solubilized collagen fibers and a method for producing the same.

### BACKGROUND ART

Solubilized collagen has excellent moisture retention properties. Compared with other bio-derived moisturizing agents such as hyaluronic acid, solubilized collagen is produced in high yield, and related products are therefore inexpensive. This point is attracting much attention, and the use of solubilized collagen as a raw material for cosmetics is highly anticipated.

Besides the aforementioned properties as a moisturizing agent, the following properties of products that contain solubilized collagen are also attracting attention. Namely, the ability to promote the adhesion and proliferation of cells, low antigenicity, high biocompatibility, and favorable biodegradability and the like.

By utilizing these properties, solubilized collagen can be used effectively in all manner of applications including cosmetics and medical materials. In terms of the use of collagen for these purposes, the use of collagen in a variety of different forms has been investigated depending on the intended application, including aqueous solutions, fibrous materials, films, sponges and gels.

It had been thought that with high-concentration solubilized collagen, the collagen should be able to be dissolved uniformly and instantaneously at the time of use. However, it was found that the collagen could not be dissolved uniformly and instantaneously in a water medium at the time of use, and that therefore obtaining the type of cosmetics that users had been anticipating was problematic.

Techniques relating to this aspect include the following.

(1) A method for producing a high-concentration solubilized collagen by using a freeze drying method as a technique for removing water.

(2) A method for producing a granulated or powdered solubilized collagen dried product (weight concentration: 95% or greater) by injecting a solubilized collagen solution (weight concentration: 3 to 10%) through a nozzle into a volatile hydrophilic organic solvent medium to form a thread-like product or film-like product within the medium, subsequently drying this thread-like or film-like product in a drying step to remove the organic solvent and water content, and then chopping or crushing the thus obtained dried product (Patent Document 1: JP 06-228505 A).

The above term “crushing” means to finely grind the product. Performing this fine grinding is difficult. Because this fine grinding cannot be achieved, when the dried product is dissolved in water, problems tend to arise, including the formation of lumps, and an inability to satisfactorily complete the dissolution operation.

(3) A method for producing a collagen molded product having excellent shape retention and environmental resistance by deaerating an aqueous collagen solution, forming a film, treating the thus obtained film with an aqueous aldehyde solution, drying the treated film, subsequently thermoforming the film into a molded product of the desired shape, and then subjecting the obtained molded product to a crosslinking treatment using an inorganic crosslinking agent and/or an organic crosslinking agent has been proposed (Patent Document 2: JP 06-200047 A). This invention employs a crosslinking treatment in obtaining the collagen molded product, and uses this crosslinking as a technique for obtaining a stabilized collagen molded product. Accordingly, there is no intention that this molded product is dissolved in water for subsequent use. Even an intention to dissolve the product in water is unreasonable from a technical standpoint.

(4) A powdered collagen produced by adjusting the pH of a collagen solution to a value in the vicinity of the isoionic point, and then subjecting the collagen solution to a spray-drying treatment as a uniform dispersion with a concentration of 3 to 10%, and a method for producing a powdered collagen by adding a basic solution to a high-concentration collagen solution to adjust the pH to a value in the vicinity of the isoionic point, subsequently adding deionized water to obtain a solution with a concentration of 3 to 10%, stirring to obtain a uniform dispersion, and then spray-drying the dispersion are also known (Patent Document 3: JP 08-27035 A). However, if this invention is viewed from the perspective of the yield of the powdered collagen that represents the product, then various problems are apparent, including poor production efficiency, the generation of lumps during dissolution, and an inability to achieve the anticipated solubility.

(5) By adding a plasticizer to a collagen dispersion using a conventional method, performing kneading and degassing, subsequently drying the dispersion to form a collagen film, and then subjecting the film to an appropriate heat treatment or ultraviolet irradiation treatment, the collagen can be imparted with hot water solubility. The heating is disclosed as being typically 50° C. to 70° C., and preferably 50° C. to 60° C. (Patent Document 4: JP 09-124804 A).

The invention described above enables the actual production of high-concentration solubilized collagen. However, using this technique to enable collagen to be dissolved uniformly and instantaneously in cold water is problematic. The use of a high-concentration solubilized collagen to obtain a water-soluble collagen is impossible.

(6) A collagen pack invention (Patent Document 5: JP 4,353,850 B, JP 2005-325056 A).

Although this invention discloses a novel collagen pack obtained using a solubilized collagen aqueous solution, it is not directly related to the use of collagen as a cosmetic.

Next, an invention was proposed for a conjugated solubilized collagen powder in which trehalose coexists within the particles (Patent Document 6: JP 2009-67703 A). In this invention, an aqueous spraying liquid containing the solubilized collagen and trehalose is prepared, and this liquid is then spray-dried to obtain a solubilized collagen powder. The trehalose coexists within the target product, and by also adding an amphoteric surfactant or a glycol compound, the hydrophilicity and the sensation of the product can be improved. However, this product does not contain solely collagen as the main component. The powder cannot be used as a cosmetic which contains collagen as the main component and undergoes uniform dissolution of the water-soluble collagen almost instantaneously at the time of use, which is the original objective for the inventors of the present invention.

With the intention of developing a product that could be dissolved uniformly and instantaneously at the time of use to prepare a water-soluble collagen, and then using this product as a cosmetic, the inventors of the present invention thought that the production of solubilized collagen fibers for the cosmetic was important, and conducted various investigations aimed at obtaining solubilized collagen fibers for cosmetics.

If the collagen can be incorporated at high concentration within an aqueous solution, then this solution can be used as a material for obtaining solubilized collagen fibers for cosmetics. Based on this thinking, the inventions described below were developed.

(1) An invention relating to a fibrous solubilized collagen cosmetic that uses a solubilized collagen solution as a raw material

Pig skin (including hair) was used as a raw material, and following completion of a hair removal liming step, the skin was treated inside a mixer drum.

The insoluble collagen was treated under alkaline conditions to produce an acidic solution of soluble collagen. The acid solution of the solubilized collagen was discharged into isopropyl alcohol from a nozzle having 1,000 discharge holes, and spun to solidify the collagen into a fibrous form. The solidified product was able to be obtained as a fiber bundle containing 1,000 continuous strands.

The product was extracted as a collagen fiber bundle immersed in alcohol, was cut to a length of approximately 1 meter, and was then hung from a stainless steel rod inside a clean bench (sterilized work apparatus) and dried (for one day and night). The folded portion where the fiber bundle was hung over the stainless steel rod was compressed by the weight of the fiber bundle, causing the fibers to adhere to each other. It was discovered that drying the fibrous solubilized collagen is difficult.

When the fibrous solubilized collagen was evaluated, it was found that not only was the raw material an acidic solution, but the fibers were very thick (60 dtx) and the rate of dissolution was extremely slow, meaning the fibers were unsuitable for obtaining a fibrous solubilized collagen cosmetic. Unfortunately, this line of thinking was abandoned. Although this line of thinking was unsuccessful, it was discovered that the method of drying a collagen fiber bundle in a state that had been immersed in a hydrophilic organic solvent such as an alcohol had a large effect on the results. This finding is utilized in the present invention.

(2) An invention relating to solubilized collagen short fibers for cosmetics

Considering the fact that a satisfactory drying operation could not be performed in the fibrous solubilized collagen cosmetic described in (1) above, it was decided to test a fibrous solubilized collagen cosmetic in the form of solubilized collagen short fibers.

An insoluble collagen was treated under alkaline conditions to produce an acidic solution of solubilized collagen. In order to improve the solubility of the solubilized collagen cosmetic that represents the product, a sodium salt of an organic acid (buffer) and sodium hydroxide were added to the acidic solution to make the solution neutral (a pH of approximately 7). Initially, sodium citrate which has a powerful buffering action was used, but because it tended to precipitate in the alcohol, this was changed to sodium lactate. Following conversion of the collagen to fibers, when the fibers were dissolved in a neutral aqueous liquid, because the isoionic point of 4.8 was not passed during dissolution, solubilized collagen fibers that exhibited good solubility were able to be obtained.

The solubilized collagen solution that had been converted to a neutral state was discharged into isopropyl alcohol from a nozzle having 1,000 discharge holes, and solidified into a fibrous form. The solidified product was able to be obtained as a fiber bundle containing 1,000 continuous strands. At the same time, the lipids dissolved in the alcohol, meaning a delipidation effect was also achieved.

A propeller was rotated immediately beneath the nozzle, so that prior to solidification as fibers, the collagen fibers were cut to form short fibers.

In order to dry the thus obtained collagen short fibers, the collagen short fibers were adhered to the surface of a stainless steel net, and were left to dry inside a clean bench. When leaving the fibers to stand inside the clean bench, it was not possible to arrange the adhered solubilized collagen short fibers in a uniform thin state having a constant thickness, and in those portions where the fibers were adhered thickly, the drying was slow, and the fibers were more likely to adhere to each other.

The content of the solubilized collagen short fibers was substantially pure collagen containing approximately 15% of water, but did also include small amounts of the sodium lactate or the like used as a buffer, and the isopropyl alcohol used as the organic solvent in the spinning bath. The fineness of the solubilized collagen fibers was approximately 20 dtx (the number of grams per 10,000 m of fiber). It was discovered that a dissolution time of approximately 30 seconds could be achieved (Patent Document 7: JP 2005-306736 A, JP 4,401,226 B).

(3) Production of solubilized collagen fibers for cosmetics (Patent Document 8: JP 2006-342472 A, JP 4,628,191 B)

The solubilized collagen fibers for cosmetics were obtained by preparing a raw material solution of solubilized collagen having an isoionic point of pH 5.0 or less, discharging the solution from a nozzle into isopropyl alcohol in the same manner as that described above, thus forming a fiber bundle of the solubilized collagen fibers for cosmetics, performing spinning and drawing of the fibers, immersing the fibers in a hydrophilic organic solvent, and then removing the water content and organic solvent from the fibers. Initially, a continuous roller drying process was adopted in which drying was performed by passing the solubilized collagen fiber bundle continuously through a winding device 21 while air was blown across the fibers (FIG. 2, left). In this case, contrary to expectations, crimping of the collagen fibers did not occur, fiber separation defects (adhesion of the fiber bundle) tended to occur, and the removal of the water content and organic solvent was unsatisfactory, resulting in poor drying efficiency, meaning a stable drying operation could not be achieved.

Next, an attempt was made to remove the water content and organic solvent by suspending the solubilized collagen fiber bundle inside a clean bench and then blowing air across the fibers. Because the solubilized collagen fiber bundle was not moved in a continuous manner, batch drying was used (this batched hanging drying is illustrated in FIG. 2, right). In this case, crimping was able to be achieved to some extent, and an improvement in fiber separation was also observed. Unfortunately, some non-uniformity in the removal of the water content and organic solvent was observed in portions of the fibers. Further, during the batch drying, adhesion of the solubilized collagen fiber bundle tended to occur in some portions. The fiber bundle was placed between two drums each having a plurality of wires embedded therein, a fiber opening (where the fiber bundle is separated into individual fibers) was performed by rotating the drums so that the wires did not make contact with each other, thus forming a fibrous state. Satisfactory fiber opening could not be achieved, and the drying treatment was inadequate, and those portions were the fibers had adhered were cut away and not used. At the time of packaging, the solubilized collagen fibers were converted to a rounded fibrous state, and subsequently collected and packaged. The thus obtained solubilized collagen fibers had a fineness of 10 dtx or less (the number of grams per 10,000 m of fiber), and were able to be dissolved uniformly in water within 30 seconds.

In this case, the amount of residual water content and the like within the fibers could not be made uniform, crimping could not be applied uniformly across the entire solubilized collagen fiber bundle, fiber separation was unable to be achieved for the entire solubilized collagen fiber bundle, the water content and organic solvent could not be removed uniformly by the drying treatment, and partial defects occurred such as adhesion of the fibers in some portions, meaning inadequacies were observed in the product. However, compared with the aforementioned case of the short fibers, the results were tolerable as a solubilized collagen for use as a cosmetic.

(4) Moisturizing agent-containing solubilized collagen fibers (Patent Document 9: JP 2008-214226 A)

Solubilized collagen fibers encapsulating a moisturizing agent that is solid at normal temperature and selected from hyaluronic acid and alginic acid. The solubilized collagen fibers are produced by preparing a solubilized collagen aqueous solution A containing the solubilized collagen having an isoionic point of pH 5.0 or less and the moisturizing agent and having a larger pH than the isoionic point of the solubilized collagen, subsequently discharging the aqueous solution into an organic solvent in a thread-like form, solidifying and spinning the solubilized collagen into a fibrous state, and then drying the fibers. Because the moisturizing agent selected from hyaluronic acid and alginic acid is encapsulated within the solubilized collagen, a product in which the solubilized collagen is the main component cannot be obtained, and therefore this invention differs from the present invention.

Conventional solubilized collagen fibers for use in cosmetics suffer the types of problems outlined above. To summarize, the specific problems are as follows.

When the collagen fiber bundle obtained following the drying step is observed, crimping has not been applied in some portions, and fiber separation defects (adhesion of the fiber bundle) exist in some portions. These problems are caused because portions exist in which the removal of the water content and the organic solvent is unsatisfactory, causing adhesion of the fibers and the like. Conventionally, those portions of the solubilized collagen fibers that are free from these problems have had to be selected for subsequent use.

In conventional solubilized collagen fibers, because the drying step does not function satisfactorily, the water content exists in a non-uniform manner within the fibers, which causes problems. In order to address this problem, the development of an invention that enables the production of a solubilized collagen fiber bundle and solubilized collagen fibers in which crimping is applied to the entire fiber bundle, fiber separation defects (where the fiber bundle undergoes adhesion) do not exist, and removal of the water content and the organic solvent occurs uniformly along the entire length direction of the solubilized collagen fibers, meaning the fibers do not adhere to one another, is very important, and the inventors considered techniques for addressing these problems.

### PRIOR ART DOCUMENTS

Patent Document 1: JP 06-228505 A

Patent Document 2: JP 06-200047 A

Patent Document 3: JP 08-27035 A

Patent Document 4: JP 09-124804 A

Patent Document 5: JP 4,353,850 B, JP 2005-325056 A

Patent Document 6: JP 2009-67703 A

Patent Document 7: JP 2005-306736 A, JP 4,401,226 B

Patent Document 8: JP 2006-342472 A, JP 4,628,191 B

Patent Document 9: JP 2008-214226 A

### DISCLOSURE OF INVENTION

### Problems Invention Aims to Solve

An object of the present invention is to provide novel solubilized collagen fibers with which solubilized collagen can be obtained by instantaneous and uniform dissolution in water at the time of use.

Specifically, an object of the present invention is to provide solubilized collagen fibers in which crimping is applied to the entire solubilized collagen fiber bundle and in which fiber separation defects (where the fiber bundle undergoes adhesion) do not exist, by obtaining solubilized collagen fibers in which the residual water content and organic solvent that remains following drying exists uniformly throughout the fibers.

### Means for Solution of the Problems

(1) As a result of intensive research, the inventors of the present invention discovered that the reason that crimping is not applied in some portions, that separation defects (where the fiber bundle undergoes adhesion) exist, and that portions in which the removal of the water content is inadequate exist within conventional solubilized collagen fibers is due to a lack of investigation of the operations wherein the solubilized collagen is subjected to a spinning and drawing step by discharging the solubilized collagen aqueous solution into an organic solvent in a thread-like form, spinning the fibers into a fiber bundle and then drawing the fibers by winding the spun solubilized collagen fiber bundle, and the drawn solubilized collagen fiber bundle is subsequently immersed in a hydrophilic organic solvent and then removed from the hydrophilic organic solvent and dried. The inventors discovered that by improving the operation of the drying step of removing the water and hydrophilic organic solvent from the solubilized collagen fiber bundle, the aforementioned object of the present invention could be achieved. Specific aspects of the present invention are described below.

(2) In conventional methods, the solubilized collagen fibers for cosmetics are suspended from a suspension device with the solubilized collagen fiber bundle still containing large amounts of water and organic solvent, and air is then blown across the fibers to vaporize the organic solvent and the water.

When air is blown across the fibers, it is very difficult to strictly control the temperature of the sterilized air at 30° C. or lower, and preferably approximately 20° C., and therefore the drying has conventionally been performed without strict controls on the heating conditions. Further, it has not been possible to completely prevent denaturation of the solubilized collagen fibers, and hanging the fibers from a suspension device has meant that the existence of non-uniformity in the amount of organic solvent and water within the fibers has been unavoidable. The portions of the solubilized collagen fibers that make contact with the suspension device, and the portions in the vicinity of the point of contact, are pulled by the weight of the solubilized collagen fibers, and therefore the fibers are pulled close together and adhere to one another upon drying, meaning they cannot be used as a cosmetic. Further, the application of the air flow to the fibers is also non-uniform, and therefore in those areas where the air flow makes inadequate contact, the fibers tend not to move, and are thus difficult to separate. It is thought that these are the reasons that crimping is not applied to some portions, that separation defects (where the fiber bundle undergoes adhesion) tend to exist, and that portions in which the removal of the water content is inadequate tend to exist.

(3) Considering the above circumstances, the inventors reached the conclusion that a drying method that pays due attention to the following points should be appropriate. Specifically, the content of the drying operation is as follows.

(a) Prior to vaporizing the organic solvent and the water content, performing a physical operation from the periphery of the solubilized collagen fiber is effective in removing preliminary amounts of the organic solvent and water. As a result, the drying operation for the organic solvent and the water content can be performed under comparatively moderate conditions. Because the weight of the solubilized collagen fiber bundle is also reduced, a more moderate operation can be used to move the solubilized collagen fiber bundle. Further, even if heat is applied from the periphery of the solubilized collagen fiber bundle, if air for which the temperature has been strictly controlled to 30° C. or lower is supplied from the periphery, then solubilized collagen fibers and a solubilized collagen fiber bundle can be obtained in which the organic solvent and the water content are distributed evenly between the inside and the outside of the solubilized collagen fiber bundle, and distributed evenly along the length direction of the solubilized collagen fiber bundle. As a result, solubilized collagen fibers can be obtained in which crimping has been applied to the entire solubilized collagen fiber bundle, fiber separation defects (where the fiber bundle undergoes adhesion) do not exist, and removal of the water content has been performed across all of the fibers.

(b) By completing an apparatus that incorporates the techniques described above, the above object can be achieved.

When subjecting the solubilized collagen fiber bundle and the solubilized collagen fibers to the removal treatment, nip rollers are installed at the supply portion where the solubilized collagen fibers are introduced to the processing operations, a portion of the water content and the organic solvent contained within the solubilized collagen fibers is squeezed from the solubilized collagen fibers along the entire length of the fibers, the solubilized collagen fiber bundle is subsequently introduced into a drying tube, sterilized air that has been controlled to a temperature of 30° C. or lower is forcibly blown through the tube to form a moving bed of air, and this moving bed of air is used to move the solubilized collagen fiber bundle for use in cosmetics, which is reduced in weight due to the removal of a portion of the water content and the organic solvent, enabling the production of solubilized collagen fibers and a solubilized collagen fiber bundle in which the organic solvent and the water content are distributed uniformly from the inside to the outside of the solubilized collagen fiber bundle, and along the length direction of the solubilized collagen fiber bundle.

(c) When the properties of the solubilized collagen fiber bundle are analyzed at the outlet of the drying tube upon completion of the drying operation, the following properties are observed.

The components contained within the solubilized collagen fibers and the amounts of those components include a solubilized collagen solid content of 66 to 87 wt %, a buffer salt content of 2 to 6 wt %, a water content of 10 to 22 wt %, and a residual hydrophilic organic solvent content of a trace amount to 6 wt % (totaling 100 wt %). The average fineness of the solubilized collagen fibers is 3 to 10 dtx, the isoionic point is 4.5 to 5.0, and the aforementioned water content of 10 to 22 wt % and the residual hydrophilic organic solvent content of a trace amount to 6 wt % exist uniformly along the length direction of the fibers.

This uniform distribution along the length of the fibers is the key to resolving the problems outline above.

The buffer salt is selected from among sodium citrate, sodium lactate and sodium phosphate.

The buffer salt is used as a pH modifier when converting the insoluble collagen to solubilized collagen, and acts effectively when dissolving the solubilized collagen fibers in water. In other words, the buffer salt is an important component in the solubilized collagen fibers of the invention.

(4) A method for producing solubilized collagen fibers that includes the drying step described above is described below.

(a) A method for producing solubilized collagen fibers that comprises: (i) a step of decomposing insoluble collagen fibers under alkaline conditions and extracting a solubilized collagen aqueous solution, and a step of performing pH modification to prepare a solubilized collagen aqueous solution that functions as a solubilized collagen fiber raw material: namely, a step of subjecting a product obtained by decomposing a skin sample containing insoluble collagen fibers under alkaline conditions to a neutralization and desalting treatment, separating the neutralized and desalted skin sample, and subsequently extracting a solubilized collagen aqueous solution having an isoionic point of pH 5.0 or less, and a step of adjusting the pH of the solubilized collagen aqueous solution in the presence of a buffer to a value within a range from 6.0 to 7.5 which is greater than the isoionic point, thus preparing a solubilized collagen aqueous solution that functions as a solubilized collagen fiber raw material; (ii) a step of subjecting the solubilized collagen aqueous solution that functions as the solubilized collagen fiber raw material to spinning and drawing to produce a solubilized collagen fiber bundle: namely, a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle obtained in (ii) to produce solubilized collagen fibers for cosmetics: namely, a step of drying the solubilized collagen fiber bundle to produce the targeted solubilized collagen fibers by passing the above solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube.

(b) A method for producing solubilized collagen fibers that further comprises a step of opening the fibers of the dried collagen fiber bundle obtained in (iii) above to form a fibrous state.

(c) The step (1) in (a) above may also be performed in the manner described below. The steps (ii) and (iii) may be the same as those described for (a).

(i) A step of decomposing insoluble collagen fibers using a protein degrading enzyme (protease) and extracting a solubilized collagen aqueous solution, and a step of performing pH modification to prepare a solubilized collagen aqueous solution that functions as a solubilized collagen fiber raw material: namely, a step of adding an alkali to a product containing collagen, obtained by decomposing a protein containing insoluble collagen using a protease and having an isoionic point of 7 to 8, to adjust the pH of the product to 9 to 10, using a carboxylic anhydride to succinylate the solubilized collagen and reduce the pH to 5 or less, subsequently precipitating and separating the solubilized collagen, and then adding an alkali in the presence of a buffer to adjust the pH to a value within a range from 6.0 to 7.5 which is greater than the isoionic point.

### Effects of the Invention

According to the present invention, a solubilized collagen fiber bundle and solubilized collagen fibers can be obtained in which crimping has been applied to the fibers, fiber separation defects (where the fiber bundle undergoes adhesion) do not exist, a portion of the water content has been removed, with the residual water content existing uniformly throughout the fibers, and fiber adhesion does not exist.

The components contained within the solubilized collagen fibers and the amounts of those components include a solubilized collagen solid content of 66 to 87 wt %, a buffer salt content of 2 to 6 wt %, a water content of 10 to 22 wt %, and a residual hydrophilic organic solvent content of a trace amount to 6 wt % (totaling 100 wt %). Solubilized collagen fibers can be obtained in which the average fineness of the solubilized collagen fibers is 3 to 10 dtx, the isoionic point is 4.5 to 5.0, and the aforementioned water content of 10 to 22 wt % and the residual hydrophilic organic solvent content of a trace amount to 6 wt % exist uniformly along the length direction of the fibers. Accordingly, it was clear that the above method for producing solubilized collagen fibers could be employed.

### BRIEF DESCRIPTION OF THE DRAWINGS

FIG. 1 is a schematic structural diagram illustrating one example of an apparatus for spinning and drawing a solubilized collagen fiber bundle for cosmetics, and then immersing the fiber bundle in a hydrophilic organic solvent in accordance with the present invention.

FIG. 2 is a diagram illustrating conventional drying devices.

FIG. 3 is a diagram illustrating a drying apparatus of the present invention.

### EMBODIMENTS OF THE INVENTION

Collagen is defined as a protein or glycoprotein having at least a partial helical structure (collagen helix). Collagen occurs as a triple helix composed of three polypeptide chains, wherein each polypeptide chain, which has a molecular weight of approximately 100,000, has a glycine residue every third position, and other frequently occurring amino acid residues include proline residues and hydroxyproline residues. Collagen is a protein that exists within all multicellular organisms, and can be extracted in large amounts from the tissues, and particularly the skin and bones, of invertebrates and vertebrates.

Due to differences in the structure of the collagen molecule, 19 different types of collagen have been reported to exist, and even for collagens classified as the same type, a plurality of different molecules may sometimes exist.

Collagen of types I, II, III and IV are mainly used as biomaterial raw materials. Type I exists in almost all connective tissue, and is the collagen type that exists in the largest quantity in living organisms. In mammals, type I collagen exists in large amounts in tendons, the dermis and bones, whereas in fish, besides the above tissues, type I collagen also exists in a large amount in the scales. In an industrial setting, collagen is often extracted from these regions.

Collagen fibers are self assemblies of the above collagen molecules, and have a specific fiber structure in which the collagen molecules are packed in series and in parallel. In an industrial setting, solubilized collagen can be obtained from the collagen fibers within tissue using an acid, an alkali or a protease.

When heat is applied to collagen, the triple helix structure of the collagen loosens, and a heat-denatured product is obtained in which each of the polypeptide chains form a random coiled shape. The temperature at which this type of structural change occurs within the collagen is termed the denaturation temperature. The denatured product is called gelatin. Compared with collagen, gelatin has a lower viscosity when converted to an aqueous solution. Further, gelatin is also known to have a high sensitivity relative to in vivo proteases.

The denaturation temperature for collagen is lowest when the collagen is in solution form. Further, collagen is generally obtained from a biological raw material, and it is said that the denaturation temperature of such biologically obtained collagen is closely related to the temperature of the living environment of the source organism. The collagen denaturation temperature in aqueous solution form is approximately 38° C. for collagen from mammals. Fish collagen generally has a lower denaturation temperature than that of mammals, and particularly in the case of cold current fish such as salmon, the temperature may fall below 20° C. in some cases. When treating collagen, performing the treatment at a temperature of 30° C. or lower, and preferably 20° C. or lower, indicates specifically that the treatment must be performed at a temperature below the denaturation temperature.

The raw material for the solubilized collagen fibers of the present invention is the collagen described above. This type of collagen is insoluble collagen, is contained within the skin tissue and other organs of animals such as cows, pigs, birds and fish, and is obtained from tissues that contain such insoluble collagen.

The inventors of the present invention initially started their research into collagen production with the aim of effectively utilizing the split leather generated as a by-product during the production of leather. This split leather can be used as the raw material.

Subsequently, as leather production shifted to tanning production methods (methods that use wet blue and wet white for leather production), split leather was no longer generated.

The tissues containing insoluble collagen described above are now used as the raw material for the purpose of producing collagen.

Examples of raw materials that can be used for the solubilized collagen fibers for cosmetics include mammal hides, and tissue derived from aquatic organisms such as fish skin and fish scales.

By selecting the raw material used for obtaining the collagen, a difference is observed in the denaturation temperature of the obtained collagen. When the raw material is in a dried state, there are no particular differences in the handling methods used, regardless of the raw material from which the solubilized collagen is derived. Currently, because of issues relating to BSE, the use of bovine-derived insoluble collagen tissue is undesirable, and the use of either porcine-derived collagen or collagen derived from an aquatic organism such as fish is preferable.

Recently, the production of collagen from synthetic peptides is garnering much attention as a potential material that suffers no danger of BSE infection. A novel polypeptide of the present invention comprises a peptide unit having an amino acid sequence represented by formula (1) shown below, and a peptide unit having an amino acid sequence represented by formula (2) shown below.

-Pro-X-Gly- (1)

-Y-Z-Gly- (2)

In these formulas, X and Z may be the same or different, and each represents Pro or Hyp, and Y represents am amino acid residue having a carboxyl group (such as Asp, Glu or Gla).

The ratio (molar ratio) between the above peptide unit (1) and the peptide unit (2) is within a range from approximately (1)/(2)=99/1 to 1/99. The polypeptide may also support apatites (see JP 4,303,137 B).

Examples of the collagen used as the raw material for the solubilized collagen fibers of the present invention include pig skin that has undergone alkali solubilization, and pig skin that has undergone enzyme solubilization and succinylation to adjust the isoionic point to an acidic value. However, the list of raw materials that can be used is not limited to the above materials, and materials produced by subjecting fish skin or fish scales to a solubilization treatment can also be used. The collagen material for use in the present invention includes materials which have an isoionic point that is sufficiently removed from the neutral region that is ideal for cosmetics, either toward the low side (acidic) or toward the high side (alkaline), and which are highly soluble in water in the neutral region but solidify within organic solvents. Provided these conditions are satisfied, synthetic collagen can also be used.

The present invention relates to solubilized collagen fibers that can be used to obtain a solubilized collagen for use as a cosmetic. Specifically, the present invention relates to the solubilized collagen fibers described below.

Namely, the solubilized collagen fibers include a solubilized collagen solid content of 66 to 87 wt %, a buffer salt content of 2 to 6 wt %, a water content of 10 to 22 wt %, and a residual hydrophilic organic solvent content of a trace amount to 6 wt % (totaling 100 wt %), wherein the average fineness of the solubilized collagen fibers is 3 to 10 dtx, the isoionic point is 4.5 to 5.0, and the aforementioned water content of 10 to 22 wt % and the residual hydrophilic organic solvent content of a trace amount to 6 wt % exist uniformly along the length direction of the fibers.

The buffer salt is selected from among sodium citrate, sodium lactate and sodium phosphate.

The solubilized collagen solid content mentioned above refers to the solid fraction composed of solubilized collagen that corresponds with the solubilized collagen contained in the solubilized collagen fiber bundle obtained by decomposing the collagen fibers to form a solubilized collagen, using the solubilized collagen to produce a solubilized collagen aqueous solution that functions as the raw material for the solubilized collagen fibers, performing spinning and drawing to form a solubilized collagen fiber bundle, and then drying the fiber bundle.

The buffer salt is a component that is added prior to the reaction for decomposing the collagen fibers for the purpose of achieving pH modification when producing the solubilized collagen aqueous solution from the solubilized collagen fiber raw material. This buffer salt has the function of ensuring instantaneous and uniform dissolution of the solubilized collagen fibers in water when the fibers are used as a cosmetic.

As described above, the buffer salt is used for the purpose of pH modification when the insoluble collagen is converted to solubilized collagen. In order to ensure rapid dissolution of the solubilized collagen fibers in aqueous solvents, dissolution must be performed within a pH region that is somewhat different from the isoionic point (pI). If the pH of the collagen or the pH of the solvent is close to the pI of the collagen, then achieving uniform dispersion and dissolution requires considerable time. In the case of an alkali-solubilized collagen, the pI is within a range from 4.5 to 5.0, and therefore a pH range that enables rapid dissolution and is ideal for cosmetic materials is from approximately 6 to 8. In order to prepare collagen fibers within this pH region, an appropriate amount of a buffer salt (such as Na lactate) is included. In order to ensure that the buffer salt is incorporated uniformly within the collagen fibers, the buffer salt is added in advance to the raw material solution that undergoes spinning, and this enables an appropriate amount of the buffer salt to be retained within the finally prepared collagen fibers.

All of the steps in the method used for decomposing the insoluble collagen fibers under alkaline conditions to obtain the solubilized collagen fibers of the present invention are described below.

The production apparatus of the present invention and all of the steps described in the present invention are conducted in an environment that is maintained in a sterilized state.

(1) A step of decomposing insoluble collagen fibers under alkaline conditions and extracting a solubilized collagen aqueous solution, and a step of performing pH modification to produce a solubilized collagen aqueous solution that functions as a solubilized collagen fiber raw material:

A step of subjecting a product obtained by decomposing a skin sample containing insoluble collagen fibers under alkaline conditions to a neutralization and desalting treatment, separating the neutralized and desalted skin sample, and subsequently extracting a solubilized collagen aqueous solution having an isoionic point of pH 5.0 or less, and a step of adjusting the pH of the solubilized collagen aqueous solution in the presence of a buffer to a value within a range from 6.0 to 7.5 which is greater than the isoionic point, thus preparing a solubilized collagen aqueous solution that functions as a solubilized collagen fiber raw material.

These steps are described below in further detail.

A method of treating insoluble collagen fibers with a material obtained by adding a small amount of an amine or an analog thereof to an aqueous solution containing both a caustic alkali and sodium sulfate (for example, see JP 46-15033 B, hereafter this method is referred to as the “alkali treatment method”) is described below.

The dermic layer is extracted from the raw hide containing the insoluble collagen that functions as the raw material, and a wet grinding mill is used to convert the dermic layer to a paste form that undergoes reaction more readily.

In the alkali treatment method, a strong alkaline composition containing approximately 4 to 5% of sodium hydroxide, approximately 10 to 12% of sodium sulfate, and approximately 1% of monomethylamine (wherein the above numbers represent weight concentrations within the solution) is used as the alkali treatment agent.

The sodium hydroxide within the strong alkaline composition decomposes the peptides of the collagen crosslinked portions (telopeptides), thereby cleaving the crosslinks and solubilizing the collagen. The sodium sulfate is used for preventing swelling of the collagen by the alkali, and preventing decomposition of the main chain portion (the triple helix portion) of the collagen. If the monomethylamine is not used, then the solubilization tends to be unsatisfactory, and a hard viscous solution (containing a large amount of multimers) is obtained.

During the solubilization treatment, it is necessary to ensure that denaturation of the collagen and precipitation of the sodium sulfate do not occur, and therefore the temperature of the solubilization treatment tank is maintained within a range from 22° C. to 27° C.

The above treatment yields a product containing an eluted solubilized collagen. By subjecting this product to a neutralization and desalting treatment, the skin that was unable to be treated is retained as neutralized and desalted skin, and this neutralized and desalted skin can be separated by a solid-liquid separation using a net-like device that allows the passage of water, such as a sieve. Alternatively, the neutralized and desalted skin can be separated by a centrifugal separation method using a low centrifugal force.

As a result of the solid-liquid separation, a solution containing solubilized collagen can be extracted. This solution is then washed to obtain the targeted solubilized collagen.

In the alkali treatment, the isoionic point of the obtained collagen is from 4.5 to 5.0. This is because the asparagine residues and glutamine residues in the collagen undergo a deamidation (releasing free ammonia) in the presence of the alkali, and are converted to aspartic acid residues and glutamic acid residues respectively.

Cosmetic items are preferably within a range from slightly acidic to neutral, and therefore during preparation of the solubilized collagen fiber raw material for use in cosmetics, no dramatic change is required in the isoionic point of the solubilized collagen. The collagen concentration is generally from approximately 3 wt % to 6 wt %.

The pH of the solubilized collagen aqueous solution is adjusted in the presence of a buffer to a value within a range from 6.0 to 7.5 which is greater than the isoionic point, thus preparing a solubilized collagen aqueous solution that functions as the solubilized collagen fiber raw material.

Following preparation of the solubilized collagen fibers, adjusting the pH of the solubilized collagen aqueous solution that functions as the solubilized collagen fiber raw material is effective in obtaining a solubilized collagen aqueous solution that can be used as a cosmetic.

The reasons for this observation are as follows.

Collagen is an amphoteric electrolyte, and has a property wherein the electric charge varies depending on the pH. The pH at which the positive and negative charges are in equilibrium, resulting in an apparent charge of zero, is the isoionic point. At this pH, the solubility of collagen deteriorates and aggregation tends to occur. Accordingly, in order to improve the solubility in the neutral region that is desirable for cosmetics, it is important that the isoionic point is somewhat removed from the neutral region. In the present invention, performing the alkali treatment alters the isoionic point to a value of 4.5 to 5.0. Alternatively, a method may be employed in which collagen having an isoionic point of approximately 7 to 8 obtained from a solubilization treatment that uses a protease enzyme is subjected to a chemical treatment such as succinylation to lower the isoionic point. The solubilization is performed to enable spinning of the obtained collagen. If the pH is held at the isoionic point, then dissolution is impossible, and therefore the solubilization must be performed on either the acidic side or the alkali side of the isoionic point. However, when solution preparation is performed on the acidic side of the isoionic point (for example, pH 3), then when an attempt is made to subsequently dissolve the obtained dried fibers in a neutral aqueous liquid (for example, pH 7) for use as a cosmetic, because the pH must pass through the isoionic point, aggregation may occur and dissolution takes a considerable length of time, meaning use as a cosmetic is problematic. On the other hand, when the solution is prepared on the alkali side of the isoionic point, and particularly at a pH within a range (pH 6.0 to 7.5) that is close to that at which final dissolution of the dried fibers occurs, the solution need not pass through the isoionic point, and the collagen remains in a readily dissociated state, meaning rapid dissolution can be achieved, and collagen fibers that are ideal for use as cosmetics can be obtained.

(2) A step of subjecting the solubilized collagen aqueous solution that functions as the solubilized collagen fiber raw material to spinning and drawing to produce a solubilized collagen fiber bundle:

A step of discharging the solubilized collagen aqueous solution obtained in (1) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent.

Specifically, this step is conducted in the manner described below.

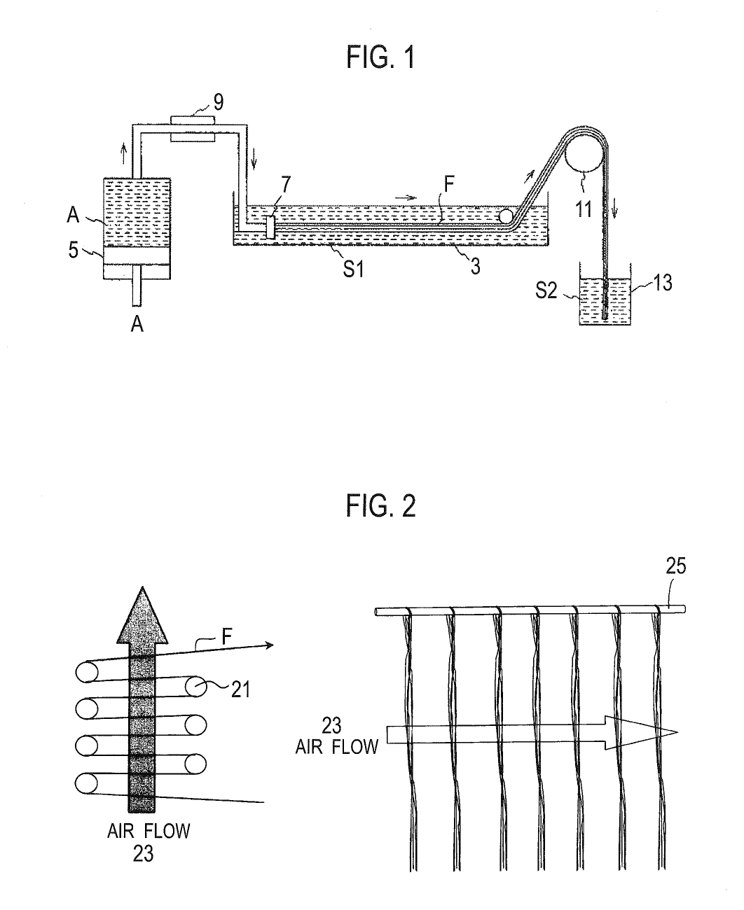

FIG. 1 is a diagram illustrating one example of a production apparatus for producing the type of solubilized collagen fibers described above.

This production apparatus 1 comprises a piston tank 5, which holds a solubilized collagen aqueous solution A and supplies the solubilized collagen aqueous solution A, a first solvent tank 3 containing an organic solvent S1 composed of isopropanol into which the supplied solubilized collagen is discharged via a nozzle 7 having a plurality of discharge holes, and in which the discharged collagen is spun and then drawn, enabling the extraction of solubilized collagen fibers containing water, a winding roller 11 which is wound at a predetermined winding rate and is used for drawing and extracting the fibers in the form of solubilized collagen fibers having a water content, and a second solvent tank 13 containing a hydrophilic organic solvent S2 into which the solubilized collagen fibers containing water that have been wound by the roller 11 are supplied.

Further, the supply of the solubilized collagen aqueous solution A from the piston tank 5 through the nozzle 7 is performed through the action of a gear pump 9. The winding roller 11 is used for winding the spun solubilized collagen fibers at a predetermined winding rate.

The piston tank 5 and the nozzle 7 are connected, via the gear pump 9, by a plastic conduit. In this example, the first solvent tank 3 has an elongated shape of a prescribed length, and the nozzle 7 is installed at one end of the first solvent tank 3 with the discharge holes directed in the horizontal direction, enabling the collagen aqueous solution discharged from the nozzle 7 to travel horizontally through the organic solvent S1 along the length of the first solvent tank 3 to the other end of the first solvent tank 3.

When the solubilized collagen aqueous solution is discharged into the organic solvent and solidified, the organic solvent used may be either a hydrophilic organic solvent or a hydrophobic organic solvent.

The solubilized collagen aqueous solution discharged into the organic solvent solidifies instantaneously to form fibers as the water diffuses within the organic solvent. In terms of facilitating the diffusion of the water contained within the collagen aqueous solution away from the fibers, a hydrophilic organic solvent is particularly suitable.

In order to enable efficient drying of the solidified fibers, the use of a solvent that enables the water to be evaporated from a state that incorporates water is desirable. In this regard, the use of a hydrophilic organic solvent is preferred. Specific examples of solvents that can be used include alcohols such as methanol, ethanol, and isopropanol, and acetone. A mixed solvent containing a plurality of different solvents may also be used. From a practical perspective, an organic solvent containing a small amount of water can also be used, and in such cases, the water content within the solvent is typically not more than approximately 15 wt % and preferably 10 wt % or less. If the water content is too high, then the collagen cannot be solidified favorably.

In the production apparatus 1 illustrated in FIG. 1, when the piston of the piston tank 5 is pressurized using compressed air, and the gear pump 9 is activated, the solubilized collagen aqueous solution A is supplied from the piston tank 5 to the nozzle 7, and is then discharged into the organic solvent S1 inside the first solvent tank 3 from the plurality of circular discharge holes in the nozzle 7.

The solubilized collagen is discharged into the organic solvent from the plurality of circular discharge holes in the nozzle 7, solidification of the solubilized collagen proceeds from the external surface toward the interior, resulting in fiber formation, and by pushing the collagen out in a horizontal direction, the plurality of collagen fibers are spun into a bundle while also undergoing a drawing treatment. The formed bundle of the solubilized collagen fibers F is pulled out of the organic solvent Si by a pulley located at the other end of the first solvent tank 3, and is then wound around a winding roller 11.

At this time, by setting the winding rate of the winding roller 11 to a faster rate than the discharge rate from the nozzle 7, the spun solubilized collagen fibers F are stretched while solidifying, and form narrow fibers having an average fineness of 10 dtx. The lower limit for the average fineness has been confirmed as low as 3 dtx.

During the period required for the collagen fibers to solidify, or specifically during the period required for the exterior of the collagen fibers to solidify, spinning and drawing of the collagen fibers is performed. During this period, the collagen fibers exist within the organic solvent, and therefore the water content within the collagen fibers is substituted with the organic solvent.

The time required for solidification varies depending on the fineness and the like of the fibers undergoing spinning. Considering such factors, the time required for solidification of the solubilized collagen fibers is generally set to approximately 8 seconds.

If a value of approximately 5 m/minute is used for the winding rate of the winding roller 11, then the length of the first solvent tank 3 in the direction of operation must be approximately 70 cm or longer.

By discharging the solubilized collagen aqueous solution through the nozzle and into the organic solvent, the solubilized collagen can be spun.

This spinning can be achieved by using a device such as a nozzle or shower head which has discharge holes that can discharge a fluid in a fiber-like form and can therefore disperse and release the fluid. The solubilized collagen aqueous solution, which has a solubilized collagen concentration from 2 to 10 wt %, and preferably from 3 to 7 wt %, is discharged into the organic solvent at a rate of 20 to 500 g/minute, and preferably 30 to 150 g/minute, through a dispersion release device having a hole diameter of approximately 0.05 to 1 mm, and preferably approximately 0.05 to 0.3 mm. As a result, solubilized collagen fibers having an average fineness of approximately 10 to 100 dtx (measured at 20° C. and 65% RH using a fineness meter) can be formed.

The thickness of the solubilized collagen fibers can be narrowed by adjusting the concentration of the discharged solubilized collagen aqueous solution, or by appropriate selection of the hole diameter of the discharge nozzle. If the concentration of the solubilized collagen aqueous solution is too low, then the spun fibers tend to rupture more easily, and a powder-like solid tends to be produced. If the nozzle hole diameter is too narrow, then the flow resistance increases, and an excessively large discharge pressure occurs at the nozzle. Consequently, the collagen fibers discharged from the nozzle in a free state undergo shrinkage in the fiber length direction during solidification, shrinking to less than approximately 0.6 times the original length and resulting in an increase in the fineness.

There is a limit to how far the fineness can be reduced using the methods of narrowing the nozzle hole diameter and reducing the concentration of the solubilized collagen aqueous solution.

In one method of resolving this problem, the collagen fibers that are spun in the solvent can be wound at a rate that is at least approximately 0.6 times the discharge rate. By using this method, the pulling force applied to the collagen fibers during spinning acts against the shrinkage in the fiber length direction, thereby drawing the fibers and enabling the preparation of fine fibers of 10 dtx or less. As a result of the drawing, the collagen fibers are collected in the hydrophilic organic solvent S2 inside the second solvent tank 13 as a fiber bundle that is free of twisting and curling.

By performing the spinning step in the organic solvent, the pig skin-derived lipids contained within the solubilized collagen aqueous solution are eluted into the organic solvent, thereby reducing the lipid content to approximately 0.1 wt % and yielding a high-purity collagen. A portion of the buffer is also eluted, but the buffer that is retained within the fibers has the effect of increasing the dissolution rate when the dried solubilized collagen fibers are dissolved in water.

If the concentration of the hydrophilic organic solvent in the first solvent tank 3 is maintained at a satisfactorily high level, then the fiber bundle need not necessarily be passed through the second solvent tank, and the fiber bundle leaving the winding roller 11 may be guided directly between nip rollers 31 and subjected to continuous spinning and drying.

In the spinning step, if the winding rate is too fast, then the fibers are prone to rupture, and therefore drawing is performed with the ratio of the winding rate relative to the discharge rate (namely, the draft) adjusted to a value of 1.5 or less.

Considering the above factors, ideal conditions for spinning collagen fibers having an average fineness of 10 dtx or less include a collagen aqueous solution concentration of 3 to 7 wt %, and preferably 3.5 to 5 wt %, a nozzle hole diameter of 0.05 to 0.18 mm, and preferably approximately 0.09 to 0.11 mm, and a draft of at 0.6 to 1.5, and preferably 1.0 to 1.2.

Each of the various conditions may be set within the respective range described above, in accordance with a formula 2 shown below.

T=100·r²cd/D Formula 2

In this formula, T represents the fineness (dtx), r represents the nozzle hole radius (mm), c represents the collagen aqueous solution concentration (wt %), d represents the collagen specific gravity (g/ml), and D represents the draft.

In terms of the types of numerical values that are actually employed, setting the various values so as to achieve a discharge rate of approximately 2 to 7 m/minute and a winding rate of approximately 2 to 10 m/minute is practical.

The wound solubilized collagen fibers are dried in a sterilized environment by air drying using sterilized air. This removes any residual water. In the case of the types of fine fibers produced in the present invention, if the fibers are in mutual contact and drying is performed in this state, then the fibers tend to adhere and bond together, forming a fibrous lump.

The reason for this observation is that because the organic solvent is removed first during drying, the residual water content contained within the solubilized collagen fibers re-dissolves the solidified collagen, and therefore as the fibers become finer, adhesion of the fibers becomes more significant.

In order to prevent this adhesion, in the present invention, the solubilized collagen fibers are immersed in a hydrophilic organic solvent prior to drying. By bringing the fibers into contact with the hydrophilic organic solvent, the water content within the collagen fibers diffuses within the organic solvent and is replaced with the organic solvent, meaning the water content of the fibers decreases and the organic solvent content increases. As a result, adhesion of the fibers during drying is reduced.

The water content of the hydrophilic organic solvent used for immersion must be low, and specifically, an organic solvent having a water content of 5 wt % or less is typically used. Specific examples of hydrophilic organic solvents that can be used include alcohols such as methanol, ethanol, and isopropanol, and acetone. A mixed solvent containing a plurality of these types of solvents may also be used. In order to avoid the retention of only water during the drying of the collagen fibers, the use of a solvent having a boiling point close to that of water, or a solvent that undergoes azeotropic distillation with water is effective, and specific examples of such solvents include ethanol and isopropanol.

When the spun solubilized collagen fibers are immersed in the hydrophilic organic solvent, the water content of the hydrophilic organic solvent increases. Once the organic solvent has been used for repeated immersion treatments, and the water content of the solvent has become excessive, the organic solvent must be replaced. Subjecting the solubilized collagen fibers to a light compression or centrifugal dewatering treatment to reduce the amount of liquid contained within the fibers immediately prior to immersion in the organic solvent is effective in reducing the frequency with which the immersion organic solvent must be replaced.

If a value of approximately 5 m/minute is used for the winding rate of the winding roller 11, then the length of the first solvent tank 3 in the direction of operation must be approximately 70 cm or longer.

(3) A step of drying the solubilized collagen fiber bundle obtained in (2) to produce solubilized collagen fibers for cosmetics

A step of drying the solubilized collagen fiber bundle to produce the targeted solubilized collagen fibers by passing the above solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower into the tube, using the moving bed of air to move and dry the solubilized collagen fiber bundle inside the tube, and then extracting the solubilized collagen fiber bundle from the tube.

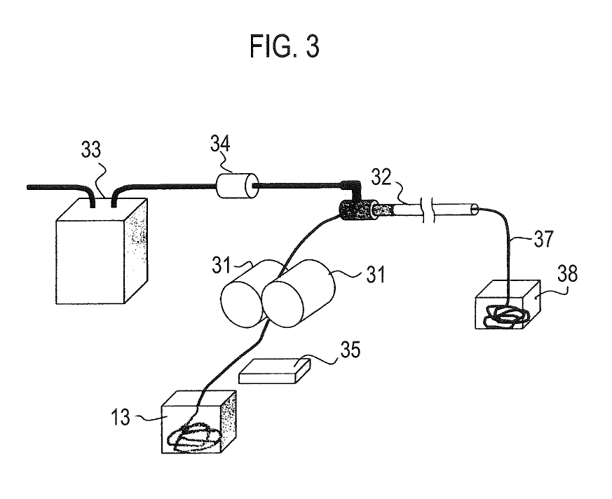

A complete drying apparatus used in the present invention for drying the spun and drawn solubilized collagen fiber bundle is illustrated in FIG. 3.

Prior to drying the spun and drawn solubilized collagen fiber bundle using air from an air supply device 33, the spun and drawn solubilized collagen fiber bundle is passed between the nip rollers 31 to squeeze a portion of the water content and alcohol content from the spun and drawn solubilized collagen fiber bundle, thereby reducing the water content and the alcohol content within the spun and drawn solubilized collagen fiber bundle. The water and alcohol that has been squeezed from the fiber bundle is collected in a liquid collection device 35. This liquid is stored in a collected liquid storage device 36 (not shown in the drawings).

Because a portion of the water and alcohol contained within the spun and drawn solubilized collagen fiber bundle can be removed prior to the drying with air, the operation of passing the spun and drawn solubilized collagen fiber bundle through the nip rollers 31 is an important pretreatment to the drying operation of drying the spun and drawn solubilized collagen fiber bundle using the air supplied from the air supply device 33.

Following squeezing of a portion of the water and alcohol contained within the spun and drawn solubilized collagen fiber bundle by passing the fiber bundle between the nip rollers 31, the solubilized collagen fibers are introduced into a drying tube (tube-shaped drying device) 32 and dried within a stream of air. The air used for the drying passes through the air supply device 33, is filtered, cleaned and sterilized by passage through a filter 34 (such as a high-efficiency particulate air filter), and in consideration of enabling stable drying of the solubilized collagen fiber bundle at a temperature of 30° C. or lower, is preferably supplied to the drying tube (tube-shaped drying device) 32 with the temperature maintained at 20° C. or lower. By supplying the air with the temperature maintained at a specific temperature or lower, the air can be distinguished from the air of the surrounding environment. The air supplied to the drying tube (tube-shaped drying device) 32 is supplied at a uniform rate. If the tube-shaped drying device 32 is constructed in the form of an aspirator, then the collagen fibers can be fed into the drying device from the suction port. Another effective method involves using a commercially available air gun designed for suctioning and transporting powders and granules (such as the air guns MAG-22S, MAG-22SV, MAG-22L and MAG-22LV manufactured by Trusco Nakayama Corporation, details of the structures of which are included in the operating manuals available from the manufacturer), and feeding the collagen fibers through the suction port.

The air is supplied at a temperature within a range from 30° C. to 0° C. If the temperature exceeds 30° C., then there is a concern that the collagen may undergo denaturation. Further, if the temperature is lower than 0° C., then the drying efficiency deteriorates.

The humidity must be 70% or less. If the humidity exceeds 70%, then the fibers are more likely to adhere. There are no disadvantages associated with a low humidity.

Movement of the solubilized collagen fiber bundle inside the drying tube (tube-shaped drying device) 32 is caused by the sterilized air having a temperature described above. The actual rate of this movement is dependent on the feed rate through the nip rollers. By appropriate control of the combination of this feed rate and the air flow rate, treatment can be conducted under ideal drying conditions (namely, conditions which not only yield favorable drying, but also result in the production of fibers with an appropriate level of crimping, and minimal adhesion and twisting of the fibers). In those cases where a polyethylene tube having a diameter of 19 mm and a length of 3 m is used, performing drying under conditions including a collagen feed rate of 2 to 3.5 m/minute and an air flow rate of 200 to 300 L/minute yields a dry solubilized collagen fiber bundle having minimal fiber adhesion and having a favorable wave applied to the entire bundle.

The change in state of the solubilized collagen fiber bundle in the drying apparatus is indicated by the following two examples of analysis results.

(1) Prior to supply to the nip rollers, the solubilized collagen fiber bundle and the like has a solid fraction concentration of 15 to 25 wt %, and a residual alcohol concentration of 70 to 80 wt %.

(2) Following passage through the nip rollers, the solubilized collagen fiber bundle and the like has a solid fraction concentration of 27 to 35 wt %, and a residual alcohol concentration of 65 to 68 wt %.

At the outlet from the drying tube, the solubilized collagen fiber bundle and the like from (1) and (2) above has a solid fraction concentration of 85 to 88 wt %, and a residual alcohol concentration of 1.0 to 6.0 wt %.

The reason for these final results is because the nip rollers operation is able to alter each of the concentrations as described above, and because conventional apparatus included no appropriate device for performing such drying, adjustment of the above concentration ranges was impossible. It can be stated that the drying method employed in this invention is very innovative.

The obtained fibers have a finished state at the tube outlet in which the solubilized collagen fiber bundle has a solid fraction concentration of 85 to 88 wt % and a residual alcohol concentration of 1.0 to 6.0 wt %, and the aforementioned water content of 10 to 22 wt % and the residual alcohol concentration of a trace amount to 6.0 wt % exist uniformly along the length direction of the fibers.

By subjecting the solubilized collagen fiber bundle at the tube outlet to a further drying operation, the residual alcohol concentration can be reduced to 0.01 wt % or less.

Because the bundle of the solubilized collagen fibers F is dried without application of a pulling load, a fiber bundle composed of crimped solubilized collagen fibers can be obtained. Moreover, by performing appropriate defibration, a fibrous state solubilized collagen can be obtained. Provided the length of the fibers is at least 2.5 cm, the fibers are intertwined, and by defibrating a fiber bundle of an appropriate length, a solubilized collagen fibrous material can be obtained.

Following completion of the drying operation, the solubilized collagen fiber bundle is subjected to fiber opening. Specifically, an opening device combining a plurality of wire drums is used to disentangle the fiber bundle, generating a fibrous state. The long solubilized collagen fibers that constitute the aforementioned solubilized collagen fiber bundle are broken up by the wire drums to form fibers having a length of 1 to 20 cm, and these fibers are entangled to form a fibrous state, so that a sheet having a uniform density is discharged, enabling collection of opened solubilized collagen fibers that can be used as solubilized collagen fibers for cosmetics.

All of the steps in the method used for decomposing the insoluble collagen fibers using a protein degrading enzyme (protease) to obtain the solubilized collagen fibers of the present invention are described below.

(1) A combination of a step of decomposing insoluble collagen fibers using a protein degrading enzyme (protease) and extracting a solubilized collagen aqueous solution, and a step of performing pH modification to prepare a solubilized collagen aqueous solution that functions as a solubilized collagen fiber raw material. Specifically, a step of decomposing a skin sample containing insoluble collagen fibers using a protein degrading enzyme (protease) and extracting an insoluble collagen aqueous solution having an isoionic point of 7 to 8, and a step of adjusting the pH in the presence of a buffer to a value within a range from 6.0 to 7.5 which is greater than the isoionic point, thus preparing a solubilized collagen aqueous solution that functions as the solubilized collagen fiber raw material.

The method using a protease is disclosed, for example, in JP 44-1175 B, and the present invention incorporates this description. Hereafter, this method is also referred to as an enzyme treatment method.

In the enzyme treatment method, approximately 1 kg of insoluble collagen fibers is prepared with a substrate concentration of approximately 2%, the pH is adjusted to 3 with lactic acid, and in those cases where an acidic protease is used as the protein degrading enzyme, treatment is performed by adding the protease in an amount of 1% relative to the substrate. The mixture is stirred at a temperature of 25° C. using a stirrer to allow the reaction to proceed.

When solubilization is performed using the enzyme treatment method, the isoionic point of the obtained collagen product is from 7 to 8. In order to collect the collagen from the obtained product, the product of the solubilization treatment is subjected to sedimentation using a centrifugal separation treatment with a large centrifugal force.

The isoionic point of the collagen is from 7 to 8, and in order to obtain a solubilized collagen raw material for use as a cosmetic, the collagen is precipitated and collected by reducing the pH to 5 or lower. In order to achieve an ideal pH for use as a cosmetic, the final pH is adjusted to a value from approximately 6.0 to 7.5. Sodium hydroxide and sodium lactate are added as a buffer.

In a solubilized collagen product obtained using a typical enzyme treatment method, a succinylation is performed to lower the isoionic point and enhance the solubility under neutral conditions, and this treatment can be used favorably in producing a solubilized collagen by this type of method.

The above step (1) can be performed in the manner described below.

(1) A step of decomposing insoluble collagen fibers using a protein degrading enzyme (protease) and extracting a solubilized collagen aqueous solution, and a step of performing pH modification to prepare a solubilized collagen aqueous solution that functions as a solubilized collagen fiber raw material.

Namely, a step of adding an alkali to a product containing collagen, obtained by decomposing a protein containing insoluble collagen using a protease and having an isoionic point of 7 to 8, to adjust the pH of the product to 9 to 10, using a carboxylic anhydride to succinylate the solubilized collagen and reduce the pH to 5 or less, and subsequently precipitating and separating the solubilized collagen. In order to then prepare a solubilized collagen aqueous solution, an alkali is added in the presence of a buffer to adjust the pH to a value within a range from 6.0 to 7.5 which is greater than the isoionic point.

(2) A step of spinning and drawing the solubilized collagen aqueous solution that functions as the solubilized collagen fibers raw material to produce a solubilized collagen fiber bundle.

Namely, a step of discharging the solubilized collagen aqueous solution obtained in (1) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent.

This step is performed using the same method as that described above for (2) in relation to the alkali treatment.

(3) A step of drying the solubilized collagen fiber bundle obtained in (2) to produce solubilized collagen fibers for cosmetics.

Namely, a step of drying the solubilized collagen fiber bundle to produce the targeted solubilized collagen fibers by passing the above solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower into the tube, using the moving bed of air to move and dry the solubilized collagen fiber bundle inside the tube, and then extracting the solubilized collagen fiber bundle from the tube.

This step is performed using the same method as that described above for (3) in relation to the alkali treatment.

In those cases where the water medium is mainly pure water, the solubilized collagen fibers dissolve readily in the pure water due to the action of the buffer incorporated within the solubilized collagen fibers. Further, by adding a small amount of an electrolyte such as an acid, base, neutral salt or buffer salt to the solubilized collagen fibers, the fibers are able to be dissolved satisfactorily within aqueous liquids. In particular, if a buffer salt such as sodium citrate, sodium lactate or sodium phosphate (namely, a salt of a weak acid and a weak base) that stabilizes the pH in a range from slightly acidic to neutral is added to the aqueous liquid to adjust the pH of the aqueous liquid to a value of approximately 5.5 to 9.0, then the solubilized collagen fibers can be more readily dissolved. The solubilized collagen fibers can be dissolved in short period of 30 seconds or less. If an excessive amount of the salt is added, then a salting-out effect makes it difficult to dissolve the collagen in aqueous liquids. The electrolyte may be incorporated within an aqueous solution.

In regard to this point, because total desalting of the solubilized collagen has not occurred following the solubilization treatment, residual electrolyte exists within the solubilized collagen. In this case, the solubilized collagen may also be used in this state, with no further modification.

Any of the various components typically added to solubilized collagen fibers for use in cosmetics may be added to the aqueous liquid, provided this addition does not impair the dissolution of the solubilized collagen in an aqueous solution. Examples of these components include moisturizing agents such as butanediol, pentanediol, glycerol, hyaluronic acid and urea, preservatives such as methyl p-hydroxybenzoate and phenoxyethanol, plant extracts such as aloe extract, alcohol-based solvents such as ethanol, ultraviolet absorbers, vitamins, anti-inflammatory agents, oils and fats such as olive oil, fatty acids, and any of the various functional components that have a specific cosmetic function.

By setting the combination ratio between the collagen fibers and the aqueous liquid so that the collagen content within the obtained cosmetic material is approximately 0.01 to 10 wt %, and particularly approximately 0.1 to 3 wt %, a uniformly dissolved cosmetic material can be obtained rapidly.

Examples of the aqueous solutions that can be used include commercially available toilet waters and cosmetic liquids.

Because of their favorable properties, the solubilized collagen fibers for cosmetics and fiber balls according to the present invention dissolve rapidly in commercially available toilet waters and cosmetic liquids. Accordingly, the user may select a toilet water or cosmetic liquid depending on individual preference, and then combine this selected water or liquid with the solubilized collagen fibers or fibrous material for cosmetic use, thus preparing a solubilized collagen solution for cosmetic use. Accordingly, a solubilized collagen cosmetic material that satisfies the needs of the user can be provided to the user in a fresh state whenever it may be required. Depending on the state of the skin of the user, a cosmetic that is suitable for that skin state can be prepared. The type of cold-temperature storage required for conventional solubilized collagen cosmetics is unnecessary, and the time required for preparing the cosmetic material is short, meaning there is no time restriction associated with use of the product, and the product may simply be used in accordance with the needs of the user.

Following dissolution, the collagen cosmetic material is prone to denaturation in a similar manner to that observed for typical collagen cosmetics in the form of aqueous solutions. However, the aforementioned treatment in which an alcohol was used as the organic solvent during the preparation of the solubilized collagen fibers has a sterilization effect on the collagen, and therefore the resulting solubilized collagen fibers, which are obtained by drying with sterilized air, are not contaminated with unwanted bacteria. Moreover, compared with collagen in a solution state, solubilized collagen in a dried state is significantly more resistant to the proliferation of bacteria or mold or the like, meaning the level of treatment required to preserve the product during transport can be reduced. Cosmetic materials that contain almost no components other than the collagen, including components such as preservatives, can be used.

Similarly, in the case of aqueous liquids for use as cosmetics, because separation is performed from collagen having a high nutritional value, the amount of added preservatives can be reduced, and the level of preservation treatment required can be reduced. Further, aqueous liquids can be sterilized more easily than collagen, and therefore by sterilizing the aqueous liquid and using aseptic packaging, the addition of preservatives becomes unnecessary.

The solubilized collagen fibers of the present invention may be marketed in the form of a fiber bundle, subjected to fiber opening and then marketed as individual fiber balls, or marketed as a combination of solubilized collagen fibers for cosmetic use and an aqueous liquid, with the fibers and the liquid provided in separated containers.

By dividing the solubilized collagen fibers into individual packages containing the amount of fibers required for a single use, measuring the amount of fibers at the time of use becomes unnecessary, and if a container containing a solubilized collagen fiber bundle or fibrous material equivalent to a single use is marked with a level indicating the amount of aqueous liquid that is required, then the user can easily measure out the amount of toilet water or the like when preparing the cosmetic material, meaning a favorable cosmetic material can be obtained on each occasion.

Further, if the aqueous liquid and the solubilized collagen fiber bundle or fibrous material are placed in a soft container having two separate compartments that are separated by a partition that can be broken by application of light force, then the solubilized collagen fibers can be mixed with and dissolved in the aqueous liquid by breaking the partition.

### EXAMPLES

The solubilized collagen fibers for cosmetics according to the present invention and the production thereof are described below in further detail, with reference to a series of examples.

### Example 1

Samples of solubilized collagen fibers for cosmetics were prepared in the manner described below, and the time required for dissolution was measured. The isoionic point of the solubilized collagen fibers was confirmed in the manner described below.

(Measurement of Isoionic Point)

A cationic exchange resin (Amberlite IPR-120B, manufactured by Organo Corporation) and an anionic exchange resin (Amberlite IPA-400, manufactured by Organo Corporation) that had been activated and washed in advance were mixed in a ratio of 2:5 to prepare a mixed bed ion exchanger. Subsequently, 100 ml of this mixed ion exchanger was brought to equilibrium using deionized water, 50 ml of a sample solution prepared with a protein concentration of 5% was added to the ion exchanger, and the mixture was held at 40° C. in a water bath and stirred gently for 30 minutes. The supernatant was then removed from the mixture, the pH of the supernatant was measured, and this measured value was used as the isoionic point (see the method described by J. W. Janus, A. W. Kenchington and A. G. Ward, Research, Vol.4, 247-248 (1951)).

(Sample 1)

Preparation of Solubilized Collagen Aqueous Solution

Using wet-salted pig hide as a raw material, liming was performed. Specifically, one half of a single wet-salted pig hide (approximately 4 kg) was cut into small skin pieces approximately 3 cm square, an amount of water equivalent to 300% of the weight of the skin pieces and 0.6% of a nonionic surfactant were added and stirred to wash the skin pieces, and the skin pieces were then recovered. Subsequently, the skin pieces were combined with an amount of water equivalent to 300% of the weight of the skin pieces, together with 0.6% of a nonionic surfactant and 0.75% of sodium carbonate, the mixture was stirred for 2 hours, and the skin pieces were once again recovered. Next, the recovered skin pieces were washed twice with amounts of water equivalent to 700% of the weight of the skin pieces, and the skin pieces were then combined with an amount of water equivalent to 300% of the weight of the skin pieces, together with 0.15% of a nonionic surfactant, 3.6% of sodium hydrosulfide, 0.84% of sodium sulfide and 2.4% of calcium hydroxide, the mixture was stirred for 16 hours, and the skin pieces were once again recovered and washed three times with amounts of water equivalent to 700% of the weight of the skin pieces.

Next, 8,000 g of an aqueous solution was prepared containing 6 wt % of sodium hydroxide, 15 wt % of sodium sulfate and 1.25 wt % of monomethylamine, and then 2,000 g of the above skin pieces (a dried wt of approximately 500 g) were added to the solution and stirred thoroughly.

The resulting mixture was held inside a sealed container at 25° C., and incubated for 5 days to solubilize the collagen. With the aqueous solution undergoing gentle stirring, an amount of sulfuric acid equal to the amount of alkali within the aqueous solution was added dropwise to neutralize the solution, thereby adjusting the pH to 4.8.

Following neutralization, the skin pieces were removed and pressed to remove any liquid contained therein, and the skin pieces were subsequently stirred for 30 minutes in approximately 8,000 g of an aqueous solution of lactic acid with a pH of 5.0, and then pressed and dewatered. This operation was repeated a further 4 times to achieve satisfactory desalting. In a neutralized state, the pH of the skin pieces is close to the isoionic point of the solubilized collagen, and therefore although the collagen is solubilized, it undergoes almost no dissolution in water even during the desalting operations, but is rather retained within the skin pieces.

The collagen content of the skin pieces following desalting was calculated from the results of measuring the total nitrogen content using the Kjeldahl method, and based on this calculated collagen content value, a sample of the desalted skin pieces equivalent to 120 g of collagen was mixed thoroughly with water and sodium lactate to obtain an aqueous solution having a collagen concentration of 4.4 wt % and a sodium lactate concentration of 1.2 wt %, thus yielding 4,000 g of a solubilized collagen aqueous solution. Subsequently, a small amount of a 20% aqueous solution of sodium hydroxide was added and stirred to adjust the pH to a value of 6.7.

Production of Solubilized Collagen Fibers

The tank 5 of a production apparatus 1 having the structure illustrated in FIG. 1 was charged with 4,000 g of the solubilized collagen aqueous solution obtained above, and 18 L of isopropanol was used as the organic solvent and placed in the first solvent tank 3 having a length of 3 m and a width of 10 cm. By operating the gear pump 9, the solubilized collagen aqueous solution was discharged into the organic solvent from the discharge holes of the nozzle 7, which were directed in the horizontal direction (hole diameter: 0.10 mm, number of holes: 1,000), at a rate of 38 g/minute (discharge rate: 4.8 m/minute). The bundle of solubilized collagen fibers that had undergone spinning within the isopropanol was pulled from the tank by the winding roller 11 at a winding rate of 5 m/minute, and was then immersed in the second solvent tank 13 which contained 5.0 L of isopropanol.

Drying of Solubilized Collagen Fibers Using a polyethylene tube having a diameter of 19 mm and a length of 3 m as the tube-shaped drying device 32, and with the feed rate of the nip rollers set to 3.5 m/minute, air at 20° C. and 55% RH was blown through the drying device at a rate of 238 L/minute.

In terms of the state of the solubilized collagen fiber bundle, the following two examples of analysis results were obtained.

(1) Prior to supply to the nip rollers, the solubilized collagen fiber bundle and the like had a solid fraction concentration of 15 to 25 wt %, and a residual alcohol concentration of 70 to 80 wt %.

(2) Following passage through the nip rollers, the solubilized collagen fiber bundle and the like had a solid fraction concentration of 27 to 35 wt %, and a residual alcohol concentration of 65 to 68 wt %.

At the outlet from the drying tube, the solubilized collagen fiber bundle and the like from (1) and (2) above had a solid fraction concentration of 85 to 88 wt %, and a residual alcohol concentration of 1.0 to 6.0 wt %.

These results mean that the nip rollers operation was able to alter each of the concentrations at the aforementioned outlet.

The obtained fibers had a finished state at the tube outlet in which the solubilized collagen fiber bundle had a solid fraction concentration of 85 to 88 wt % and a residual alcohol concentration of 1.0 to 6.0 wt %, and the aforementioned water content of 10 to 22 wt % and the residual alcohol concentration of a trace amount to 6.0 wt % existed uniformly along the length direction of the fibers.

The solubilized collagen fiber bundle and the like at the tube outlet had a solid fraction concentration of 82.1 wt % and a residual alcohol concentration of 4.8 wt %.

A 50 g bundle of solubilized collagen fibers having an average fineness of 3.7 dtx (excluding the 10 in at either end of the fibers) and a natural level of crimping was obtained (isoionic point: pH 4.9). Almost no adhered fiber portions were observed. The fineness was measured using a fineness meter (Deniel Computer DC-11A, manufactured by Search Co., Ltd.), by measuring 20 fibers from each sample in an environment at 20° C. and 65% RH, and then calculating the average fineness value (this method was also used in example 2 onward). The pH of a 0.5 wt % solution prepared by dissolving the solubilized collagen fibers in deionized water was 7.1.

The above solubilized collagen fibers were composed of 79 wt % of solubilized collagen, 2.3 wt % of sodium lactate, 4.8 wt % of isopropyl alcohol, and 13.9 wt % of water (totaling 100 wt %). Further, measurement of the lipid content within the solubilized collagen fibers using the “oil and fat content” hexane extraction method prescribed in JIS K6503: (2001) 5.6 yielded a result of less than 0.1 wt %.

Approximately 10 mg of the obtained solubilized collagen fibers were placed in the palm of the hand, 1 mL of water was added, and when mixing was performed with the index finger, the fibers dissolved in approximately 30 seconds, forming a state that was usable as a cosmetic material.

### Example 2

(Sample 2) With the exception of altering only the nip rollers feed rate to 2 m/minute, the spun solubilized collagen fibers of the sample 1 (that were immersed in the second solvent tank 13) were dried under the same conditions as those described for the example 1.

The measurement results for the solubilized collagen fiber bundle at each of the steps within the drying treatment were as follows.

Prior to supply to the nip rollers, the solubilized collagen fiber bundle and the like had a solid fraction concentration of 20 wt %, and a residual alcohol concentration of 74 wt %.

Following passage through the nip rollers, the solubilized collagen fiber bundle and the like had a solid fraction concentration of 30 wt %, and a residual alcohol concentration of 66 wt %.

At the outlet from the drying tube, the solubilized collagen fiber bundle and the like had a solid fraction concentration of 87.1 wt %, and a residual alcohol concentration of 1.5 wt %.

A 50 g bundle of solubilized collagen fibers having an average fineness of 3.7 dtx (excluding the 10 m at either end of the fibers) and a natural level of crimping was obtained (isoionic point: pH 4.9). Almost no adhered fiber portions were observed. The solubilized collagen fibers were composed of 84 wt % of solubilized collagen, 2.5 wt % of sodium lactate, 1.5 wt % of isopropyl alcohol, and 12.0 wt % of water (totaling 100 wt %). The oil and fat content, measured using the hexane extraction method, was less than 0.1 wt %.

Approximately 10 mg (approximately 3 cm) of the obtained solubilized collagen fiber bundle was cut with scissors and placed in the palm of the hand, 1 mL of water was added, and when mixing was performed with the index finger, the fibers dissolved in approximately 30 seconds, forming a state that was usable as a cosmetic material.

### Example 3

(Sample 3)

The solubilized collagen fiber bundle of the sample 2 was subjected to fiber opening using an opening device.

From the resulting fibrous-like solubilized collagen fiber sheet, 10 mg of the solubilized collagen fibers were placed in the palm of the hand, 1 mL of water was added, and when mixing was performed with the index finger, the fibers dissolved in approximately 20 seconds, forming a state that was usable as a cosmetic material.

### Comparative Example 1

(Sample 4)

The spun solubilized collagen fibers of the sample 1 (that were immersed in the second solvent tank 13) were dried using a conventional roller-type drying device illustrated on the left side of FIG. 2. The distance between the left and right rollers was 1.8 m, five stages were employed (drying path total length: 9 m), and air at 25° C. and 45% RH was blown through the device at a rate of 0.5 in/second.

A 50 g bundle of solubilized collagen fibers having an average fineness of 3.7 dtx (excluding the 10 m at either end of the fibers) was obtained (isoionic point: pH 4.9). The fibers had a linear form with absolutely no crimping, and a high degree of fiber adhesion was very noticeable.

The solubilized collagen fibers were composed of 77.8 wt % of solubilized collagen, 2.7 wt % of sodium lactate, 5.2 wt % of isopropyl alcohol, and 14.3 wt % of water (totaling 100 wt %). The oil and fat content, measured using the hexane extraction method, was less than 0.1 wt %.

Approximately 10 mg of the obtained solubilized collagen fibers were placed in the palm of the hand, 1 mL of water was added, and mixing was performed with the index finger, but even after 60 seconds the fibers had not completely dissolved, and a state that was usable as a cosmetic material could not be obtained. This was because the adhered portions of the fibers did not dissolve.

### Comparative Example 2

(Sample 5)

The spun solubilized collagen fibers of the sample 1 (that were immersed in the second solvent tank 13) were cut to a length of 1.2 m, and then dried using a conventional hanging-type batch drying device illustrated on the right side of FIG. 2. The spun solubilized collagen fibers were lightly stroked with the fingers to squeeze out the alcohol, were subsequently hung over a stainless steel bar installed inside a clean bench, and were then dried under conditions of 20° C. and 45% RH. The air flow was generated by the exhaust flow from the clean bench.

A 50 g bundle of solubilized collagen fibers having an average fineness of 3.7 dtx (excluding the adhered portions) was obtained (isoionic point: pH 4.9). The solubilized collagen fibers were composed of 81.7 wt % of solubilized collagen, 2.9 wt % of sodium lactate, 3.5 wt % of isopropyl alcohol, and 11.9 wt % of water (totaling 100 wt %). The oil and fat content, measured using the hexane extraction method, was less than 0.1 wt %.

The portions of the fibers within the vicinity of the contact with the stainless steel bar adhered to one another to form a bent U-shape. Fiber adhesion was also noticed within other portions of the fibers. With the exception of these adhered portions, the level of crimping was comparatively favorable.

Approximately 10 mg of the obtained solubilized collagen fibers were placed in the palm of the hand, 1 mL of water was added, and mixing was performed with the index finger, but even after 60 seconds the fibers had not completely dissolved, and a state that was usable as a cosmetic material could not be obtained. This was because the adhered portions of the fibers did not dissolve.

### Example 4

A 10 g sample was taken from each of the solubilized collagen fibers obtained in example 1, example 2, comparative example 1 and comparative example 2, portions in which the fibers had adhered (namely, portions where the fibers could not be separated into individual fibers, but rather formed an adhered lump) were cut away, the weight of these adhered portions was measured, and the proportion of adhered portions relative to the total weight of the solubilized collagen fibers was calculated. The results were as follows.

Example 1: 1% or less

Example 2: 1% or less

Comparative example 1: 50%

Comparative example 2: 20%

It is evident that the drying method of the present invention is extremely effective in preventing fiber adhesion.

### Example 5

(Sample 6)

Preparation of Solubilized Collagen Aqueous Solution

A wet-salted pig hide was cut into pieces and limed in the same manner as that described for the sample 1. The thus obtained skin pieces were fed through a chopper having a hole diameter of 16 mm, and then converted to a paste form using a grinding mill (Masscolloider, manufactured by Masuko Sangyo Co., Ltd.). The paste-like pig skin was subjected to a delipidation treatment using ethanol, and was then dried. A 100 g sample was taken from the dried product, 1,900 g of deionized water was added, and hydrochloric acid was added to adjust the pH to 3.0 while the mixture was stirred with a mixer. Subsequently, 20 g of an acidic protease formulation (Denapsin 2P, manufactured by Nagase ChemteX Corporation) was added, and stirring was continued for 24 hours at 25° C. to solubilize the collagen. The thus obtained solubilized collagen aqueous solution was adjusted to a pH of 9 to 10 by adding 2N sodium hydroxide, 40 g of succinic anhydride was dissolved in acetone and added, and with the temperature held at 10° C. and the pH maintained between 9 and 10, reaction (succinylation) was performed for 2 hours. Following completion of the reaction, hydrochloric acid was used to adjust the pH of the reaction solution to 4.5, thus precipitating the collagen. The precipitate was collected by performing a centrifugal separation at 3,000 G for 10 minutes, and the precipitate was then washed with ethanol and dried, yielding a succinylated solubilized collagen dried product. To a 60 g sample of this dried product were added 29 g of sodium lactate and 1,920 g of water, and the mixture was stirred to obtain a solubilized collagen aqueous solution having a collagen concentration of 4.5 wt % (pH: 6.8, sodium lactate concentration: 1.2 wt %).

Production of Solubilized Collagen Fibers

Using the above solubilized collagen aqueous solution, solubilized collagen fibers were produced using the same apparatus and the same operations as those described for sample 1.

The measurement results for the solubilized collagen fiber bundle at each of the steps within the drying treatment were as follows.

Prior to supply to the nip rollers, the solubilized collagen fiber bundle and the like had a solid fraction concentration of 21 wt %, and a residual alcohol concentration of 76 wt %.

Following passage through the nip rollers, the solubilized collagen fiber bundle and the like had a solid fraction concentration of 32 wt %, and a residual alcohol concentration of 64 wt %.

At the outlet from the drying tube, the solubilized collagen fiber bundle and the like had a solid fraction concentration of 84.6 wt %, and a residual alcohol concentration of 3.0 wt %.

A 50 g bundle of solubilized collagen fibers having an average fineness of 4.1 dtx (excluding the 10 m at either end of the fibers) and a natural level of crimping was obtained (isoionic point: pH 4.5). The solubilized collagen fibers were composed of 83 wt % of solubilized collagen, 3.2 wt % of sodium lactate, 2.8 wt % of isopropyl alcohol, and 11.0 wt % of water (totaling 100 wt %).

Approximately 10 mg of the obtained solubilized collagen fiber bundle was placed in the palm of the hand, 1 mL of water was added, and when mixing was performed with the index finger, the fibers dissolved in approximately 30 seconds, forming a state that was usable as a cosmetic material.

### INDUSTRIAL APPLICABILITY

In terms of producing solubilized collagen, dissolving the solubilized collagen rapidly and uniformly, and then using the resulting product, the present invention enables novel trends within the fields of foodstuffs and pharmaceuticals to be used as commercial products.

### DESCRIPTION OF REFERENCE SIGNS

1 Production apparatus

3 First solvent tank

5 Piston tank

7 Nozzle

9 Gear pump

11 Winding roller

13 Second solvent tank

S1 Organic solvent

S2 Hydrophilic organic solvent

A Solubilized collagen aqueous solution

F Solubilized collagen fiber bundle

21 Roller

23 Air flow

25 Hanging device

31 Nip roller

32 Tube-shaped drying device

33 Air supply device

34 Filter

35 Liquid collection device

36 Collected liquid storage device (not shown in the drawings)

37 Solubilized collagen fibers for cosmetics

38 Collection unit for solubilized collagen fibers for cosmetics

1. Solubilized collagen fibers, comprising a solubilized collagen solid content of 66 to 87 wt %, a buffer salt content of 2 to 6 wt %, a water content of 10 to 22 wt %, and a residual hydrophilic organic solvent content of a trace amount to 6 wt % (totaling 100 wt %), wherein an average fineness of the solubilized collagen fibers is 3 to 10 dtx, an isoionic point is 4.5 to 5.0, and the water content of 10 to 22 wt % and the residual hydrophilic organic solvent content of a trace amount to 6 wt % exist uniformly along a length direction of the fibers.

2. The solubilized collagen fibers according to claim 1, wherein the buffer salt is selected from among sodium citrate, sodium lactate and sodium phosphate.

3. The solubilized collagen fibers according to claim 1 or 2, obtained by a method for producing solubilized collagen fibers comprising (i) a step of subjecting a product obtained by decomposing a skin sample containing insoluble collagen fibers under alkaline conditions to a neutralization and desalting treatment, separating a neutralized and desalted skin sample, and subsequently extracting a solubilized collagen aqueous solution having an isoionic point of pH 5.0 or less, and a step of adjusting pH of the solubilized collagen aqueous solution in presence of a buffer salt to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in the step (i) into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle from the step (ii) to produce targeted solubilized collagen fibers by passing the solubilized collagen fiber bundle through nip rollers, introducing a thus obtained solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube.

4. Fibrous state solubilized collagen fibers, obtained by subjecting the solubilized collagen fibers according to claim 3 to fiber opening.

5. The solubilized collagen fibers according to claim 1 or 2, obtained by a method for producing solubilized collagen fibers comprising (i) a step of decomposing a skin sample containing insoluble collagen fibers insoluble collagen fibers using a protein degrading enzyme (protease) and extracting a solubilized collagen aqueous solution having an isoionic point of 7 to 8, and a step of adding an alkali to the solubilized collagen aqueous solution to adjust pH to 9 to 10, using a carboxylic anhydride to succinylate the solubilized collagen and reduce an isoionic point to a pH of 5 or less, subsequently precipitating and separating the solubilized collagen, and then adding an alkali in presence of a buffer salt to adjust p1-1 to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in the step (i) into an organic solvent in a thread-like form, spinning the solubilized collagen into a fiber bundle, drawing the spun solubilized collagen fiber bundle by winding, and then immersing the solubilized collagen fiber bundle in a hydrophilic organic solvent; and (iii) a step of drying the solubilized collagen fiber bundle from the step (ii) to produce targeted solubilized collagen fibers by passing the solubilized collagen fiber bundle through nip rollers, introducing a thus obtained solubilized collagen fiber bundle having a reduced water content and a reduced concentration of the hydrophilic organic solvent into a drying tube, forming a moving bed of air inside the tube by blowing sterilized air having a temperature of 30° C. or lower into the tube, using the moving bed of air to move and dry the solubilized collagen fiber bundle inside the tube, and then extracting the solubilized collagen fiber bundle from the tube.

6. Fibrous state solubilized collagen fibers, obtained by subjecting the solubilized collagen fibers according to claim 5 to fiber opening.

7. A method for producing a solubilized collagen aqueous solution, the method comprising dissolving the solubilized collagen fibers according to any one of claims 1 to 6 in an aqueous solution to obtain a solubilized collagen aqueous solution.
